(19) United States
(12) Patent Application Publication
(10) Pub. No.: US 2024/0398484 A1
(43) Pub. Date: Dec. 5, 2024

(54) MEASURED POSITIONING SURGICAL NAVIGATION SYSTEM AND POSITIONING SURGICAL NAVIGATION METHOD

(71) Applicant: Central South University, Changsha (CN)

(72) Inventors: Li XIONG, Changsha (CN); Jiangjie ZHANG, Changsha (CN); Piao ZHANG, Changsha (CN)

(73) Assignee: Central South University, Changsha, HN (CN)

(21) Appl. No.: 18/031,741

(22) PCT Filed: Jun. 8, 2022
(86) PCT No.: PCT/CN2022/097645

§ 371 Date: Apr. 13, 2023

(30) Foreign Application Priority Data
Dec. 31, 2021 (CN) 202111657291.8

(51) Int. Cl. A61B 34/20

(52) U.S. Cl. CPC A61B 34/20; A61B 2034/2051; A61B 2034/2063

(57) ABSTRACT

The present disclosure provides a measured positioning surgical navigation system and method. The system includes: a three-dimensional (3D) image reconstruction module, including a ct device, a magnetic resonance device and an ultrasound device; a 3D positioning error correction module, including a plurality of base stations and positioning chips; a scalpel, provided with a tail position sensor; and a controller, wherein the devices, the base stations, the positioning chips, and the sensor are each communicatively connected to the controller. A first 3D image is established according to the devices of the 3D image reconstruction module, a second 3D image of a lesion of a patient is constructed according to the 3D positioning error correction module, and fusion correction is performed on the two 3D images to obtain a target positioning data set, and in combination with a real-time position of the scalpel, surgical step navigation is performed, thereby improving the surgery.

### CROSS REFERENCE TO RELATED APPLICATIONS

This application claims the benefit of Chinese patent application No. 202111657291.8, filed on Dec. 31, 2021, the contents of which are incorporated herein by reference.

### FIELD OF THE INVENTION

Embodiments of the present disclosure relate to the technical field of medical informatics, in particular to a measured positioning surgical navigation system and a positioning surgical navigation method.

### BACKGROUND OF THE INVENTION

Currently, with the rapid development of medical imaging, CT and MRI can achieve accurate scanning of extremely thin layers, surgeons can not only know the positions of important structures from these imaging data, but also three-dimensional reconstruction can be conducted to further understand the spatial position relationship of blood vessels of adjacent organs, however, the prior art cannot achieve fully automatic surgical navigation, since the blood vessels of the human body are branched from deep to superficial, that is, large arterial vessels that supply blood and large venous vessels that recover blood are mostly in the deep, and protected, but our surgical approach is usually from the outside to the inside, and from the shallow to the deep to ligate superficial vessels step by step until main supply vessels and main collecting vessels in the deep are finally ligated. This results in the problem of continuously encountering bleeding during surgery, so humans have invented various hemostatic devices. However, these hemostasis operations are aimed at secondary blood vessels, which means that a lot of unnecessary bleeding occurs. The main reason is the lack of advanced control of superior main supply vessels in deep parenchyma.

It can be seen that there is an urgent need for an efficient and accurate measured positioning surgical navigation system.

### SUMMARY OF THE INVENTION

In view of this, the embodiments of the present disclosure provide a measured positioning surgical navigation system and a positioning surgical navigation method, which at least partially solves the problems in the prior art that surgery is difficult to perform and surgery efficiency and precision are poor.

In a first aspect, an embodiment of the present disclosure provides a measured positioning surgical navigation system, including:

a three-dimensional image reconstruction module, including a ct device, a magnetic resonance device, and an ultrasound device;

a three-dimensional positioning error correction module, including a plurality of base stations and a plurality of positioning chips, wherein a plurality of target points of an operating room and a plurality of target points of a ct room are each provided with one of the base stations, and all of the positioning chips are arranged around a labeled position of a patient;

a scalpel, wherein a position of a tail of the scalpel is provided with a sensor, and real-time position data of a tip of the scalpel is calculated according to real-time position data of the sensor; and

a controller, wherein the ct device, the magnetic resonance device, the ultrasound device, the base stations, the positioning chips, and the sensor are each communicatively connected to the controller, the controller is configured to control the ct device, the magnetic resonance device and the ultrasound device to acquire and integrate two-dimensional image data of a patient to obtain a first 3D image, the controller controls all of the base stations to conduct communication matching with all of the positioning chips to establish a second 3D image, and by using communication data of the three-dimensional positioning error correction module as core data, positioning error correction is conducted to obtain a target positioning data set, and the scalpel is controlled to perform surgical steps in conjunction with the real-time position data of the tip of the scalpel.

According to a specific implementation of the embodiment of the present disclosure, the three-dimensional image reconstruction module further includes a 3D scanner and an X-ray instrument, and the 3D scanner and the X-ray instrument are each communicatively connected to the controller.

According to a specific implementation of the embodiment of the present disclosure, the controller is connected with a cloud computing server.

According to a specific implementation of the embodiment of the present disclosure, among all of the positioning chips, one positioning chip is configured to locate position information of the lesion in cooperation with the base stations, and the remaining positioning chips are arranged around different labeled points of the lesion, respectively, and are configured to construct a three-dimensional image of the body of the patient in cooperation with the base stations.

In a second aspect, an embodiment of the present disclosure provides a positioning surgical navigation method, wherein the method is applied to any one of the measured positioning surgical navigation systems in the above-mentioned first aspect, and includes:

providing a measured positioning surgical navigation system, the measured positioning surgical navigation system including a three-dimensional image reconstruction module, a three-dimensional positioning error correction module, a scalpel, and a controller;

acquiring a plurality of 2D examination images of a patient through a ct device, a magnetic resonance device and an ultrasound device of the three-dimensional image reconstruction module, respectively, and integrating all of the examination images to construct a first 3D image corresponding to a focal region of the patient;

respectively disposing a plurality of base stations of the three-dimensional positioning error correction module on each target point of an operating room and each target point of a ct room, implanting a plurality of positioning chips of the three-dimensional positioning error correction module around a labeled position in the body of the patient through puncture, and constructing a second 3D image through communication matching of the positioning chips with the base stations;

uploading the first 3D image and the second 3D image to the controller for fusion adjustment using communication data of the three-dimensional positioning error correction module as core data to obtain a target positioning data set; and

when a surgery is performed, acquiring, by the controller, a position of a sensor on the scalpel in real time to calculate a real-time position of a tip of the scalpel, and determining whether a position of the tip of the scalpel is offset according to the target positioning data set.

According to a specific implementation of the embodiment of the present disclosure, the step of determining the position of the tip of the scalpel is offset according to the target positioning data set includes:

detecting whether the real-time position of the tip of the scalpel belongs to the target positioning data set;

if yes, determining that the position of the tip of the scalpel is not offset; and

if not, determining that the position of the tip of the scalpel is offset.

The measured positioning surgical navigation solution in the embodiment of the present disclosure includes: the three-dimensional image reconstruction module, including the ct device, the magnetic resonance device, and the ultrasound device; the three-dimensional positioning error correction module, including the plurality of the base stations and the plurality of the positioning chips, wherein the plurality of the target points of the operating room and the plurality of the target points of the ct room are each provided with one of the base stations, and all of the positioning chips are arranged around the labeled position of the patient; the scalpel, wherein the position of the tail of the scalpel is provided with the sensor, and real-time position data of the tip of the scalpel is calculated according to real-time position data of the sensor; and the controller, wherein the ct device, the magnetic resonance device, the ultrasound device, the base stations, the positioning chips, and the sensor are each communicatively connected to the controller, the controller is configured to control the ct device, the magnetic resonance device and the ultrasound device to acquire and integrate two-dimensional image data of the patient to obtain the first 3D image, the controller controls all of the base stations to conduct communication matching with all of the positioning chips to establish the second 3D image, and by using communication data of the three-dimensional positioning error correction module as core data, positioning error correction is conducted to obtain the target positioning data set, and the scalpel is controlled to perform the surgical steps in conjunction with the real-time position data of the tip of the scalpel.

The beneficial effects of the embodiments of the present disclosure are as follows: by means of the solution of the present disclosure, the first 3D image of the patient is established according to the devices of the three-dimensional image reconstruction module, respectively, and the second 3D image of the lesion of the patient is constructed according to the communication matching of the three-dimensional positioning error correction module, and then fusion correction is performed on the first 3D image and the second 3D image to obtain the target positioning data set, and in combination with the real-time position of the scalpel, surgical step navigation is performed, thereby improving the safety, efficiency and precision of the surgery.

### BRIEF DESCRIPTION OF DRAWINGS

In order to illustrate the technical solutions of the embodiments of the present disclosure more clearly, the drawings required to be used in the embodiments will be briefly described below, and it is obvious that the drawings in the following description are only some embodiments of the present disclosure, and other drawings can be obtained from these drawings for those of ordinary skill in the art without paying inventive step.

FIG. 1 is a structural schematic diagram of a measured positioning surgical navigation system according to an embodiment of the present disclosure;

FIG. 2 is a schematic diagram of disposing positions of positioning chips according to an embodiment of the present disclosure;

FIG. 3 is a schematic flowchart of a positioning surgical navigation method according to an embodiment of the present disclosure;

FIG. 4 is a schematic flowchart of positioning error correction involved in the positioning surgical navigation method according to the embodiment of the present disclosure;

FIG. 5 is a schematic diagram of specific implementation steps involved in the positioning surgical navigation method according to the embodiment of the present disclosure.

### DETAILED DESCRIPTION OF THE EMBODIMENTS

The embodiments of the present disclosure are described in detail below in conjunction with the accompanying drawings.

The embodiments of the present disclosure are described below with reference to specific examples, and those skilled in the art can easily understand other advantages and effects of the present disclosure from the contents disclosed in the description. Obviously, the described embodiments are only a part of the embodiments of the present disclosure, but not all of the embodiments. The present disclosure can also be implemented or applied in other different embodiments, and various details in the description can be variously modified or changed based on different views and applications without departing from the spirit of the present disclosure. It should be noted that the following embodiments and features in the embodiments may be combined with each other without conflict. Based on the embodiments in the present disclosure, all other embodiments obtained by those of ordinary skill in the art without making inventive labor belong to the scope of protection of the present disclosure.

It should be noted that various aspects of the embodiments within the scope of the appended claims are described below. It should be apparent that aspects described herein may be embodied in a wide variety of forms and that any specific structure and/or function described herein is merely illustrative. Based on the present disclosure, one skilled in the art should appreciate that one aspect described herein may be implemented independently of any other aspects and that two or more of these aspects may be combined in various ways. For example, an apparatus may be implemented and/or a method may be practiced using any number of the aspects set forth herein. In addition, the apparatus may be implemented and/or the method may be practiced using other structures and/or functions other than one or more of the aspects described herein.

It should also be noted that the drawings provided in the following embodiments illustrate only schematically the basic idea of the present disclosure, the drawings show only components related to the present disclosure, and are not drawn in terms of the number, shape and size of the components in actual implementation, the type, number and scale of the components in the actual implementation may be varied arbitrarily, and the layout of the components may be more complex.

In addition, in the following description, specific details are provided to facilitate a thorough understanding of the examples. However, it will be understood by those skilled in the art that the aspects may be practiced without these specific details.

From a medical point of view, on one hand, medical imaging has developed rapidly at present, CT and MRI can achieve accurate scanning of extremely thin layers, and surgeons can not only know the positions of important structures from these imaging data, but also three-dimensional reconstruction can be conducted to further understand the spatial position relationship of blood vessels of adjacent organs to guide surgery. However, it is still impossible to achieve complete robotic surgical resection at present. In addition to secondary reasons such as respiratory movement, the main reason is that a scalpel cannot locate its own position, and therefore, correct movement instructions cannot be given to the scalpel. Therefore, a scalpel tip positioning system is developed.

On the other hand, as previously mentioned, not only the positioning of the scalpel, but also the intraoperative movement of organs increases the difficulty of positioning. Therefore, it is envisaged that by pre-operative positioning and implanting positioning chips into important positions of a surgical area, position information is fed back in real time, and in combination with the scalpel tip positioning system, the surgery is navigated accurately and quickly in real time.

In addition, the blood vessels of the human body are branched step by step from deep to superficial, that is, large arterial vessels that supply blood and large venous vessels that recover blood are mostly in the deep, and protected, but our surgical approach is usually from the outside to the inside, and from the shallow to the deep to ligate superficial vessels step by step until main supply vessels and main collecting vessels in the deep are finally ligated. This results in the problem of continuously encountering bleeding during surgery, so humans have invented various hemostatic devices. However, these hemostasis operations are aimed at secondary blood vessels, which means that a lot of unnecessary bleeding occurs. The main reason is the lack of advanced control of superior main supply vessels in deep parenchyma.

Further, taking liver cancer as an example: it is one of the most common malignant tumors with the second death rate in China, which seriously threatens the life and health of our people. Surgical treatment is an important means for patients with liver cancer to obtain a long-term survival rate, wherein hepatectomy is the first choice for radical treatment, and its main difficulties are to control surgical bleeding and reduce postoperative complications. Since the liver has double blood supply (including two sets of blood supply systems of portal vein and hepatic artery) and hepatic venous return systems, the structure of an intrahepatic ductal system is complex and irregular, blood supply is very abundant (a total blood flow accounts for about ¼ of a cardiac output, and can be up to 1500 ml/min), and during the amputation process, it is very easy to cause massive haemorrhage, postoperative liver failure, and other surgical side injuries, which can directly lead to death in severe cases. In addition, tumor metastasis caused by intraoperative compression through blood vessels is also one of the reasons for postoperative tumor recurrence and metastasis.

An embodiment of the present disclosure provides a measured positioning surgical navigation system, which can be applied to a surgical guidance process in medical scenarios.

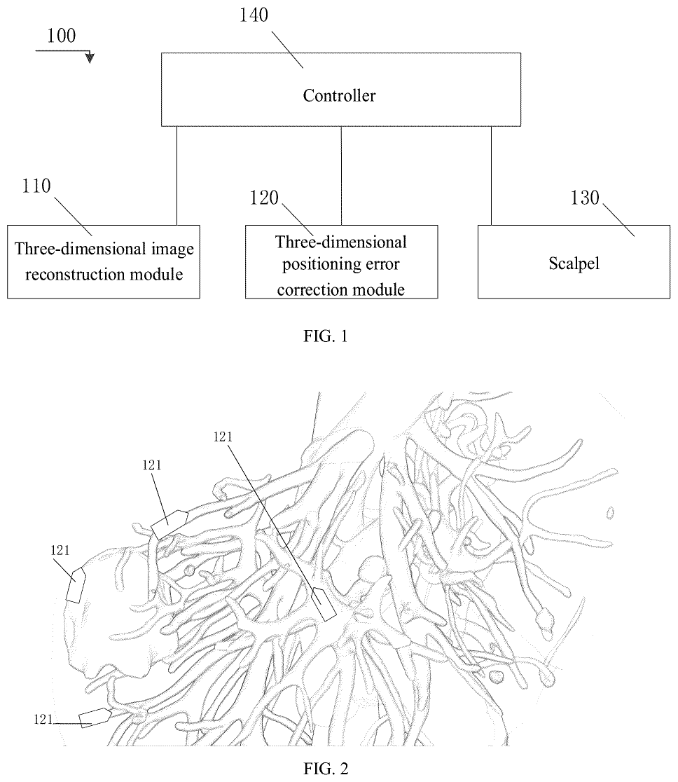

Referring to FIG. 1, a structural schematic diagram of a measured positioning surgical navigation system according to an embodiment of the present disclosure is shown. As shown in FIG. 1, the measured positioning surgical navigation system 100 mainly includes:

a three-dimensional image reconstruction module 110, including a ct device, a magnetic resonance device, and an ultrasound device;

a three-dimensional positioning error correction module 120, including a plurality of base stations and a plurality of positioning chips 121, wherein a plurality of target points of an operating room and a plurality of target points of a ct room are each provided with one of the base stations, and all of the positioning chips 121 are arranged around a labeled position of a patient;

a scalpel 130, wherein a position of a tail of the scalpel 130 is provided with a sensor, and real-time position data of a tip of the scalpel 130 is calculated according to real-time position data of the sensor; and

a controller 140, wherein the ct device, the magnetic resonance device, the ultrasound device, the base stations, the positioning chips 121, and the sensor are each communicatively connected to the controller 140, the controller 140 is configured to control the ct device, the magnetic resonance device and the ultrasound device to acquire and integrate two-dimensional image data of a patient to obtain a first 3D image, the controller 140 controls all of the base stations to conduct communication matching with all of the positioning chips 121 to establish a second 3D image, and by using communication data of the three-dimensional positioning error correction module 120 as core data, positioning error correction is conducted to obtain a target positioning data set, and the scalpel 130 is controlled to perform surgical steps in conjunction with the real-time position data of the tip of the scalpel 130.

During specific assembly, three fixed positions of the operating room and three fixed positions of the ct room may each be provided with one of the base stations. Of course, a solution mode of adding a reference object to adjust a base station module to a mobile detachable base station module can also be adopted. Meanwhile, considering that the position of the tip of the scalpel needs to be detected in real time during the surgery, however, the tip needs to be operated accordingly during the surgery, and is accompanied by heat, and it is not possible to provide a positioning device directly at the position of the tip for positioning, the position of the tail of the scalpel 130 may be provided with the sensor such as a gyroscope, and the position of the tip can be inversely deduced by collecting the position of the tail through the sensor. After the base stations are placed at the fixed positions, all of the positioning chips 121 are implanted into the patient through puncture, and then the ct device, the magnetic resonance device, the ultrasound device, the base stations, the positioning chips 121, and the sensor are each communicatively connected to the controller 140. Of course, the number of the base stations and the number of the positioning chips 121 can be set according to specific needs, which are not limited here.

In use, the controller 140 may control the ct device, the magnetic resonance device, and the ultrasound device to acquire and integrate image data of the patient to obtain the first 3D image, considering that the second 3D image is a still image, and there will be accompanied by movement of the patient's body and the change in the location of a surgical site during the surgery, all of the base stations may be controlled by the controller 140 to conduct communication matching with all of the positioning chips 121 to establish the second 3D image, and by using communication data of the three-dimensional positioning error correction module 120 as core data, positioning error correction is conducted to obtain the target positioning data set, and the scalpel 130 is controlled to perform surgical steps in conjunction with the real-time position data of the tip of the scalpel.

According to the measured positioning surgical navigation system provided by this embodiment, by utilizing CT, magnetic resonance, a 3D reconstruction system, a novel scalpel tip positioning navigation system, a chip-base station module communication system and other technologies, a plurality of sets of three-dimensional image views are modelled by collecting pictures and surrounding environment data, and through real-time position information delivered by a chip-base station module, the deviation information of the three-dimensional image views is accurately calculated in conjunction with the relevant number theory formula, the correction reduces errors and high-frequency real-time refreshing 3D image positioning views are reconstructed to achieve medical applications of integrating the real-time 3D views into surgical navigation at a lower price, assisting and guiding operators or intelligent surgical robots to perform fine mechanical operations such as navigation surgery.

On the basis of the above embodiment, the three-dimensional image reconstruction module 110 further includes a 3D scanner and an X-ray instrument, and the 3D scanner and the X-ray instrument are each communicatively connected to the controller 140.

In specific implementation, the three-dimensional image reconstruction module 110 may further include the 3D scanner and the X-ray machine, and the 3D scanner and the X-ray apparatus are each communicatively connected to the controller 140, so that the constructed first 3D image has higher accuracy, avoiding too much calculation of subsequent error correction to reduce the efficiency. Of course, other penetrating 3D imaging devices may be added as needed, which will not be listed here.

Optionally, the controller 140 is connected with a cloud computing server.

In specific implementation, the controller 140 may be connected to the cloud computing server to increase the computational efficiency in view of a large amount of data of the three-dimensional images, which will reduce the processing efficiency by simply performing computing using a processor of an electronic device.

Further, among all of the positioning chips 121, one positioning chip 121 is configured to locate position information of the lesion in cooperation with the base stations, and the remaining positioning chips 121 are arranged around different labeled points of the lesion, respectively, and are configured to construct a three-dimensional image of the body of the patient in cooperation with the base stations.

In specific implementation, as shown in FIG. 2, there is a tumor in the liver of the patient, considering that while constructing the three-dimensional image of the lesion of the patient, positioning is also required to be conducted in conjunction with the external environment, one positioning chip 121 may be configured to locate the position information of the lesion in cooperation with the base stations, the remaining positioning chips 121 may be arranged around different labeled points of the lesion, respectively, and may be configured to construct the three-dimensional image of the body of the patient in cooperation with the base stations. Of course, the number of the positioning chips 121 configured to locate the position information of the lesion in cooperation with the base stations may also be increased according to actual requirements such as the need to locate a specific area or the need for higher positioning accuracy.

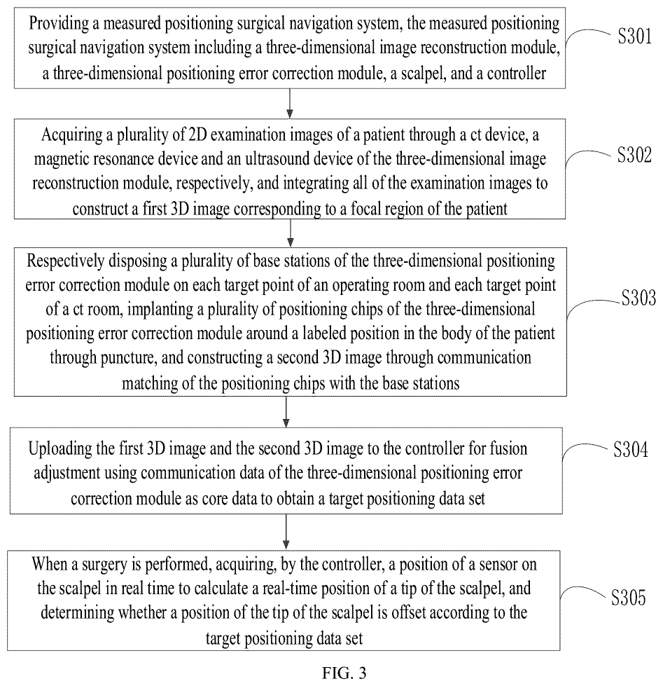

Corresponding to the system embodiment above, referring to FIG. 3, an embodiment of the present disclosure further provides a positioning surgical navigation method, including:

S301, providing a measured positioning surgical navigation system, the measured positioning surgical navigation system including a three-dimensional image reconstruction module, a three-dimensional positioning error correction module, a scalpel, and a controller;

S302, acquiring a plurality of 2D examination images of a patient through a ct device, a magnetic resonance device and an ultrasound device of the three-dimensional image reconstruction module, respectively, and integrating all of the examination images to construct a first 3D image corresponding to a focal region of the patient;

in specific implementation, the patient needs to be subjected to relevant preoperative examination before surgery, and the ct device, the magnetic resonance device and the ultrasound device of the three-dimensional image reconstruction module are used to acquire the plurality of the 2D examination images of the patient, respectively, and all of the examination images are integrated to construct the first 3D image corresponding to the focal region of the patient.

S303, Respectively disposing a plurality of base stations of the three-dimensional positioning error correction module on each target point of an operating room and each target point of a ct room, implanting a plurality of positioning chips of the three-dimensional positioning error correction module around a labeled position in the body of the patient through puncture, and constructing a second 3D image through communication matching of the positioning chips with the base stations;

in specific implementation, after obtaining the first 3D image, six base stations of the three-dimensional positioning error correction module may be further arranged on three target points of the operating room and three target points of the ct room, respectively, four positioning chips of the three-dimensional positioning error correction module are implanted around the lesion through puncture, and through the communication matching of the positioning chips with the base stations, after receiving the data, the base station module can pre-construct a 3D image system of the body of the patient, constructing the second 3D image.

S304, Uploading the first 3D image and the second 3D image to the controller for fusion adjustment using communication data of the three-dimensional positioning error correction module as core data to obtain a target positioning data set;

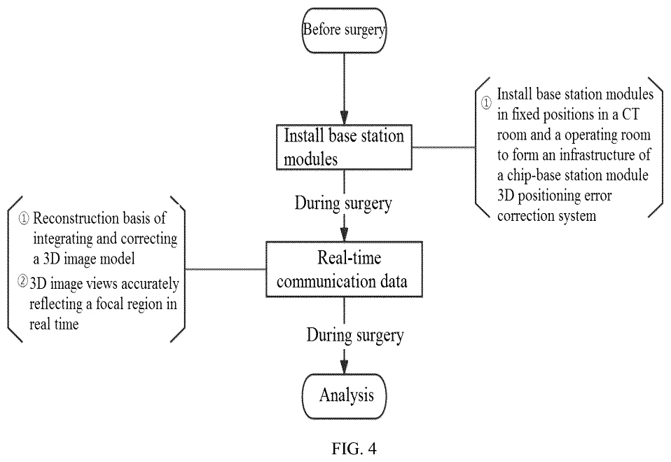

in specific implementation, as shown in FIG. 4, after obtaining the first 3D image and the second 3D image, the first 3D image and the second 3D image may be uploaded to the controller for fusion adjustment using the communication data of the three-dimensional positioning error correction module as the core data to obtain the target positioning data set, for example, a big data algorithm is used to perform fusion adjustment on a plurality of sets of 3D image data, and by using the data obtained by the three-dimensional positioning error correction module as core data, error correction is performed to obtain the target positioning data set, so as to realize real-time accurate feedback of the surgical operation or the blood flow resistance control operation of the surgical navigation system.

S305, When a surgery is performed, acquiring, by the controller, a position of a sensor on the scalpel in real time to calculate a real-time position of a tip of the scalpel, and determining whether a position of the tip of the scalpel is offset according to the target positioning data set.

On the basis of the above embodiment, the step S305 of determining whether the position of the tip of the scalpel is offset according to the target positioning data set includes:

detecting whether the real-time position of the tip of the scalpel belongs to the target positioning data set;

if yes, determining that the position of the tip of the scalpel is not offset; and

if not, determining that the position of the tip of the scalpel is offset.

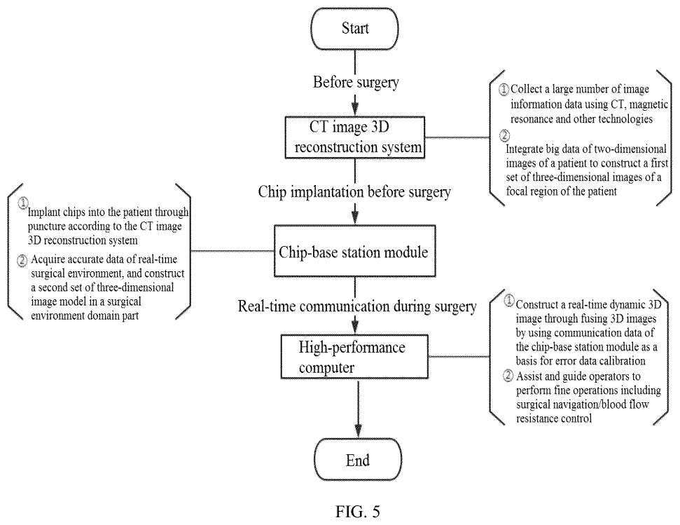

In specific implementation, when the controller controls the scalpel to perform surgery on the patient, the sensor may be arranged on the tail of the scalpel to facilitate detecting position data of the tip of the scalpel in real time, the controller may then match the position data with the target positioning data set, it is determined whether the real-time position of the tip of the scalpel belongs to the target positioning data set, if the real-time position of the tip of the scalpel belongs to the target positioning data set, it can be determined that the position of the tip of the scalpel is not offset, if the real-time position of the tip of the scalpel does not belong to the target positioning data set, it can be determined that the position of the tip of the scalpel is offset, and a prompt message can be sent to remind operators to conduct treatment timely, thus avoiding medical accidents, and improving the safety of the surgery. The implementation flow of the positioning surgical navigation method is shown in FIG. 5.

In summary, according to the measured positioning surgical navigation system and the positioning surgical navigation method provided by this embodiment, by providing the three-dimensional image reconstruction module, the three-dimensional positioning error correction module, the scalpel, and the controller, the first 3D image is constructed by acquiring two-dimensional images of the patient, respectively, and the second 3D image is constructed by data interaction of the positioning chips and the base stations, and then fusion correction is performed on the first 3D image and the second 3D image to obtain the more accurate target positioning data set for the controller to control the scalpel to perform surgical operation navigation, thereby improving the safety, efficiency and precision of the surgery.

The above are only specific embodiments of the present disclosure, but the protection scope of the present disclosure is not limited thereto, and any variations or replacements that can be easily thought of by those skilled in the art within the technical scope of the present disclosure should be covered within the protection scope of the present disclosure. Therefore, the protection scope of the present disclosure should be subject to the protection scope of the claims.

1. A measured positioning surgical navigation system, comprising:
a three-dimensional image reconstruction module, comprising a ct device, a magnetic resonance device, and an ultrasound device;
a three-dimensional positioning error correction module, comprising a plurality of base stations and a plurality of positioning chips, wherein a plurality of target points of an operating room and a plurality of target points of a ct room are each provided with one of the base stations, and all of the positioning chips are arranged around a lesion labeling position in a patient through puncture;
a scalpel, wherein a position of a tail of the scalpel is provided with a sensor, and real-time position data of a tip of the scalpel is obtained through an inverse method according to real-time position data of the sensor; and
a controller, wherein the ct device, the magnetic resonance device, the ultrasound device, the base stations, the positioning chips, and the sensor are each communicatively connected to the controller, the controller is configured to control the ct device, the magnetic resonance device and the ultrasound device to acquire and integrate two-dimensional image data of a patient to obtain a first 3D image, the controller controls all of the base stations to conduct communication matching with all of the positioning chips to establish a second 3D image, and by using communication data of real-time position information delivered by a chip-base station module of the three-dimensional positioning error correction module as core data, positioning error correction is conducted in conjunction with a number theory formula to obtain a target positioning data set, and the scalpel is controlled to perform surgical steps in conjunction with the real-time position data of the tip of the scalpel, wherein the communication data is obtained according to a real-time communication matching process in which the base stations send signals, the signals are received by the positioning chips for signal feedback, and then the base stations receive the signal feedback.

2. The system according to claim 1, wherein the three-dimensional image reconstruction module further comprises a 3D scanner and an X-ray instrument, and the 3D scanner and the X-ray instrument are each communicatively connected to the controller.

3. The system according to claim 1, wherein the controller is connected with a cloud computing server.

4. The system according to claim 1, wherein among all of the positioning chips, one positioning chip is configured to locate position information of the labeled position in cooperation with the base stations, and the remaining positioning chips are arranged around different labeled points of the labeled position, respectively, and are configured to construct a three-dimensional image of the body of the patient in cooperation with the base stations.

5. A positioning surgical navigation method, wherein the method is applied to the measured positioning surgical navigation system according to claim 1, and comprises:
acquiring a plurality of 2D examination images of a patient through the ct device, the magnetic resonance device and the ultrasound device of the three-dimensional image reconstruction module, respectively, and integrating all of the examination images to construct a first 3D image corresponding to a focal region of the patient;
respectively disposing the plurality of the base stations of the three-dimensional positioning error correction module on each target point of an operating room and each target point of a ct room, implanting the plurality of the positioning chips of the three-dimensional positioning error correction module around a labeled position in the body of the patient through puncture, and constructing a second 3D image through communication matching of the positioning chips with the base stations;
uploading the first 3D image and the second 3D image to the controller for fusion adjustment using communication data of the three-dimensional positioning error correction module as core data to obtain a target positioning data set; and
when a surgery is performed, acquiring, by the controller, a position of the sensor on the scalpel in real time to calculate a real-time position of a tip of the scalpel, and determining whether a position of the tip of the scalpel is offset according to the target positioning data set.

6. The method according to claim 5, wherein the step of determining whether the position of the tip of the scalpel is offset according to the target positioning data set comprises:
detecting whether the real-time position of the tip of the scalpel belongs to the target positioning data set;
if yes, determining that the position of the tip of the scalpel is not offset; and
if not, determining that the position of the tip of the scalpel is offset.
